(19) United States
(12) Patent Application Publication
(10) Pub. No.: US 2026/0351395 A1
(43) Pub. Date: Nov. 5, 2026

(54) PERSONALIZED THRESHOLDS FOR VITAL SIGN MONITORING

(71) Applicant: University of Virginia Patent Foundation, Charlottesville, VA (US)

(72) Inventors: Margaret Kramer Sande, Charlottesville, VA (US); William Adu-Jamfi, Lynchburg, VA (US); Jesse Thanouphet Srioudom, Brambleton, VA (US)

(21) Appl. No.: 19/664,631

(22) Filed: Apr. 30, 2026

Related U.S. Application Data
(60) Provisional application No. 63/797,603, filed on Apr. 30, 2025.

(51) Int. Cl. G16H 40/20; G16H 10/60; G16H 40/63; G16H 50/30

(52) U.S. Cl. CPC G16H 40/20; G16H 10/60; G16H 40/63; G16H 50/30

(57) ABSTRACT

A physiological monitoring system generates personalized alarm thresholds for patient physiological signs using patient-specific data and a trained neural network model. The system receives patient-specific data including medical history data, medication information, demographic data, and prior physiological sign measurements, preprocesses the patient-specific data, and inputs the preprocessed data into a feedforward neural network. The feedforward neural network generates patient-specific expected values and variability parameters for one or more physiological signs, including heart rate, oxygen saturation, systolic blood pressure, and diastolic blood pressure. A threshold generation module computes individualized risk zones from the expected values and variability parameters, and a zone classification module classifies incoming physiological sign measurements relative to the individualized risk zones. An alert detection module analyzes temporal sequences of classifications and generates a clinical notification when repeated abnormal classifications or escalation among risk zones indicates potential patient deterioration.

### APPLICATION CROSS-REFERENCE

The present Application claims the benefit of U.S. Provisional Patent Application No. 63/797,603 filed on 30 Apr. 2025, the entirety of which incorporated herein by reference.

### COMPUTER PROGRAM LISTING APPENDIX

A computer program listing appendix, consisting of one file, is submitted herewith in ASCII plain text format. The file is titled 20260430UVA0027US2CODE.txt, created on 30 Apr. 2026 with a size of 17,228 bytes. The material contained in the computer program listing appendix is hereby incorporated by reference in its entirety.

### BACKGROUND

Emergency departments (EDs) across the healthcare system are increasingly burdened by excessive patient volume, leading to significant delays in treatment and care. Long wait times in overcrowded EDs not only degrade the overall quality of patient care but also contribute to preventable deterioration of patient health, especially for individuals whose conditions may worsen without timely intervention. The dual impact of high patient acuity and constrained medical resources often leaves healthcare providers overwhelmed and unable to maintain adequate patient surveillance, particularly for those in waiting areas.

A critical shortcoming in current systems is the lack of continuous, real-time monitoring for patients prior to being formally admitted. Traditional vital sign monitoring devices often require manual input and lack integration with centralized systems capable of dynamic, patient-specific alerting. Patients waiting in holding areas may deteriorate without trained hospital staff aware of the deterioration or a need to act.

Furthermore, data transmission is hindered by proprietary wireless communication protocols, and a general lack of interconnectedness or open protocols ultimately restricting direct access to physiological data for external analysis or dashboard integration. These limitations have impeded the development of comprehensive central monitoring solutions for non-admitted patients.

Efforts to design portable and simplified monitoring hardware have generally failed to yield novel hardware innovations, with limited scope for real-time data streaming or interoperability with existing infrastructure. Consequently, innovation in this domain has increasingly shifted toward leveraging advanced software solutions, particularly predictive modeling using deep learning, to anticipate adverse health events or threshold crossings based on individualized physiological trends. Such predictive systems offer the potential to enhance triage and resource allocation in the ED, providing critical insights to healthcare providers when physical monitoring is not feasible.

There remains a need for an intelligent, patient-tailored monitoring solution that enables real-time prediction of vital sign thresholds, facilitating timely alerts and improved patient safety during extended wait periods in overcrowded emergency departments and other acute care environments.

### SUMMARY

A software aimed at transforming how patients are observed in emergency department (ED) waiting rooms and beyond is disclosed herein. One of our key innovations is a multi-output regression model (Python, Feedforward Neural Network) that generates personalized thresholds for each patient. Rather than using static, population-based cutoffs, our algorithm adjusts alarm thresholds dynamically based on a patient's age, gender, weight, and baseline vitals—aiming to reduce alarm fatigue, improve early detection of deterioration, and provide more tailored care.

The disclosed material provides methods and systems for improving patient monitoring and alerting by machine learning techniques and seamless integration with healthcare telemetry and electronic record systems. A method is described for generating personalized alarm thresholds for patient vital signs by incorporating patient-specific data, including medical history, medication information, demographic data, and prior vital sign measurements, into a Feedforward Neural Network. The thresholds are dynamically adjusted to enhance accuracy and personalization, enabling classification of vital signs into risk zones for improved monitoring.

Additionally, a method for predicting patient deterioration is disclosed, utilizing time-series data of vital signs to train a Neural Network model capable of identifying trends and generating alerts when the likelihood of deterioration exceeds predefined thresholds. A system is also provided for validating these personalized thresholds and trend algorithms using real-time, de-identified patient data, with clinician feedback incorporated to optimize sensitivity and specificity.

The disclosure includes a method for integrating these personalized alarm thresholds and trend prediction algorithms with hospital Electronic Health Record (EHR) systems, such as EPIC, to enable seamless monitoring workflows. This integration facilitates real-time alerts, bidirectional communication for continuous updates, and a user interface for intuitive display of risk zones and predictions to clinicians. Together, these innovations aim to enhance patient safety, improve clinical workflows, and optimize healthcare delivery.

In some aspects, the disclosed technology relates to a physiological monitoring system including one or more physiological sensors configured to generate physiological sign measurements for a patient, one or more processors communicatively coupled to the one or more physiological sensors, and one or more non-transitory computer-readable media storing instructions executable by the one or more processors. The instructions cause the physiological monitoring system to receive patient-specific data for the patient, preprocess the patient-specific data to generate a patient-specific input vector, apply the patient-specific input vector to a trained neural network model, and generate, using the trained neural network model, patient-specific expected values and corresponding patient-specific variability parameters for a plurality of physiological signs. The physiological monitoring system generates individualized risk zones for each physiological sign based on the patient-specific expected values and the corresponding patient-specific variability parameters, where the variability parameters define patient-specific tolerance ranges. The physiological monitoring system classifies the physiological sign measurements relative to the individualized risk zones to generate a temporal sequence of classifications, detects a temporal pattern in the temporal sequence of classifications indicative of physiological deterioration, and generates a clinical notification in response to detecting the temporal pattern. The physiological monitoring system thereby improves real-time monitoring by dynamically adapting threshold ranges based on patient-specific variability.

In some embodiments, the one or more physiological sensors include at least one of an electrocardiogram sensor, a pulse oximeter, a digital thermometer, a blood pressure sensor, a digital stethoscope, a wearable patch sensor, or an optical photoplethysmography sensor. In some embodiments, the physiological monitoring system is configured for use in an emergency department waiting room. In some embodiments, the clinical notification is generated by activating a display alert, an audible alarm, an electronic message, an automated telephone notification, or a combination thereof. In some embodiments, the physiological monitoring system is integrated with an Electronic Health Record system through bidirectional communication that updates the patient-specific data, the individualized risk zones, or both.

In some embodiments, the individualized risk zones are defined based on a plurality of ranges derived from the patient-specific variability parameters. The individualized risk zones may include ranges defined by one or more standard deviation-based multipliers applied to the patient-specific variability parameters. Different physiological signs of the plurality of physiological signs may be associated with different variability-based threshold definitions. In some embodiments, the physiological sign measurements are processed in real time. In some embodiments, the trained neural network model is configured to simultaneously output the patient-specific expected values and the patient-specific variability parameters in a single inference pass. In some embodiments, the clinical notification is generated only when a plurality of consecutive measurements satisfy an alert condition.

In some aspects, the disclosed technology relates to a non-transitory computer-readable medium storing instructions that, when executed by one or more processors, cause the one or more processors to perform operations. The operations include receiving patient-specific data for a patient from an Electronic Health Record system and one or more physiological monitoring devices, encoding and scaling the patient-specific data to generate a patient-specific input vector, processing the patient-specific input vector using a trained multi-output feedforward neural network, and generating predicted expected values and predicted variability parameters for one or more physiological signs. The operations further include computing personalized threshold ranges for the one or more physiological signs using the predicted expected values and the predicted variability parameters, where the variability parameters define patient-specific tolerance ranges. The operations further include assigning incoming physiological sign measurements to risk zones defined by the personalized threshold ranges to generate a temporal sequence of classifications, detecting a temporal alert condition based on the temporal sequence of classifications, and outputting an alert in response to detecting the temporal alert condition. The operations improve monitoring accuracy by dynamically adapting threshold ranges to patient-specific variability.

In some embodiments, the temporal alert condition includes a three-measurement sliding window. In some embodiments, the temporal alert condition includes a yellow-to-yellow-to-red progression. In some embodiments, the temporal alert condition includes a green-to-yellow-to-red progression. In some embodiments, the operations further include updating or validating the trained multi-output feedforward neural network based on validation loss, clinician feedback, additional de-identified patient data, or a combination thereof.

In some aspects, the disclosed technology relates to a computer-implemented method for personalized physiological monitoring of a patient. The method includes receiving, by one or more processors, patient-specific data for the patient, the patient-specific data including demographic data and baseline physiological sign measurements, and preprocessing, by the one or more processors, the patient-specific data to generate a patient-specific input vector. The method further includes applying, by the one or more processors, the patient-specific input vector to a trained neural network model and generating, by the trained neural network model, patient-specific expected values and patient-specific variability parameters for a plurality of physiological signs. The method further includes generating, by the one or more processors, individualized risk zones for each physiological sign of the plurality of physiological signs based on a patient-specific expected value and a corresponding patient-specific variability parameter, where the variability parameters define patient-specific tolerance ranges. The method further includes receiving, by the one or more processors, physiological sign measurements for the patient from one or more physiological sensors, classifying, by the one or more processors, each physiological sign measurement of the physiological sign measurements relative to the individualized risk zones to generate a temporal sequence of zone classifications, detecting a temporal pattern in the temporal sequence of zone classifications indicative of physiological deterioration, and generating, by the one or more processors, a clinical notification in response to detecting an alert condition within the temporal pattern.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

A better understanding of features and advantages of the present disclosure will be obtained by reference to the following detailed description, which sets forth illustrative embodiments of the disclosure, and the accompanying drawings.

FIG. 1 illustrates a high-level schematic of an embodiment of a neural network model disclosed herein.

FIG. 2 provides a flow chart of an embodiment of a neural network model disclosed herein.

FIG. 3 illustrates a validation process for a neural network model configured to generate personalized vital sign thresholds.

FIG. 4 illustrates patient information for a patient case study generated from the disclosed neural network model.

FIG. 5 illustrates output information for a patient case study generated by the disclosed neural network model.

FIG. 6 illustrates the alerting aspect of the patient case study generated using the disclosed neural network model.

FIG. 7 illustrates pulse measurements over time for a patient case study generated from the disclosed neural network model.

FIG. 8 illustrates oxygen saturation measurements over time for a patient case study generated from the disclosed neural network model.

### DETAILED DESCRIPTION

The present disclosure generally relates to patient monitoring and alerting, and more particularly to techniques for generating personalized alarm thresholds for patient vital signs using patient-specific data that may include medical history data comprising a problem list, medication information, demographic data, and prior vital sign measurements. In some embodiments, a Feedforward Neural Network or other Neural Network model may be used to generate personalized alarm thresholds, classify patient vital signs into risk zones including normal, caution, and critical zones, predict patient deterioration from vital sign trends based on time-series data, validate generated thresholds and predictions using continuous real-time, de-identified patient data and clinician feedback, and support integration with hospital Electronic Health Record systems, such as EPIC, for use in patient monitoring workflows in emergency departments, including ED waiting rooms and beyond.

Provided herein is a neural network model trained to predict personalized vital sign thresholds (heart rate, oxygen saturation, systolic and diastolic blood pressure) based on patient demographics and prior measurements. The training dataset in an example embodiment includes the Human Vital Signs Dataset from Kaggle, which includes 200,000 samples with key physiological parameters such as heart rate, respiratory rate, body temperature, oxygen saturation, blood pressure, and patient demographics like age, gender, weight, and height.

Prior to describing the disclosure in deeper detail, the following terms may be helpful.

As used herein the terms “computer,” “computer system,” “computer module,” and the like refer to a system that may include all the necessary electronics, software, memory, storage, databases, firmware, logic/state machines, microprocessors, communication links, displays or other visual or audio user interfaces, printing devices, and any other input/output interfaces to perform the functions described herein and/or to achieve the results described herein. Systems may include at least one processor and system memory/data storage structures, which may include random access memory (RAM) and read-only memory (ROM). The at least one processor may include one or more conventional microprocessors and one or more supplementary co-processors such as math co-processors, field-programmable-gate arrays, neural engines, or the like. The data storage structures discussed herein may include an appropriate combination of magnetic, optical and/or semiconductor memory, and may include, for example, RAM, ROM, flash drive, an optical disc such as a compact disc and/or a hard disk or drive.

Additionally, a software application that adapts a controller to perform the methods disclosed herein may be read into a main memory of the at least one processor from a computer-readable medium. The term “computer-readable medium”, as used herein, refers to any medium that provides or participates in providing instructions to the at least one processor of a computer system (or any other processor of a device described herein) for execution. Such a medium may take many forms, including but not limited to, non-volatile media and volatile media. Non-volatile media include, for example, optical, magnetic, or opto-magnetic disks, such as memory. Volatile media include dynamic random access memory (DRAM), which typically constitutes the main memory. Common forms of computer-readable media include, for example, a floppy disk, a flexible disk, hard disk, magnetic tape, any other magnetic medium, a CD-ROM, DVD, any other optical medium, a RAM, a PROM, an EPROM or EEPROM (electronically erasable programmable read-only memory), a FLASH-EEPROM, any other memory chip or cartridge, or any other medium from which a computer can read.

While in embodiments, the execution of sequences of instructions in the software application causes at least one processor to perform the methods/processes described herein, hard-wired circuitry may be used in place of, or in combination with, software instructions for implementation of the methods/processes of the present invention. Therefore, embodiments of the present disclosure are not limited to any specific combination of hardware and/or software.

Certain aspects of the present disclosure may provide for an application, the application may provide for an interface, e.g., a web-based user interface or other type of network interface to provide known patient and medical record data and parameters. That is, multiple users may assist in training neural network embodiments by providing data entered into the network for training, calibration, and/or validation purposes.

As used herein a “feed forward neural network” (“FFNN”) is an artificial neural network where information moves only in one direction: forward. Data travels from input nodes, through any hidden nodes, and finally to output nodes. Unlike recurrent networks, there are no cycles or loops in the network. Main components of an FFNN can include: An input layer which receives the raw data or features, such as inputs from sensors and/or patient information and/or medical history and passes that information into the network for processing. Between the input layer and an output layer are one or more hidden layers, which are intermediate layers where the learning process occurs. Within the hidden layers, each node applies a mathematical transformation to the incoming data so that the network can identify patterns and relationships.

The behavior of the network is shaped by weights and biases, which are parameters adjusted during training to reduce prediction errors. The weights determine the strength of the connection between neurons, while the biases allow an activation function to shift. The activation function serves as a mathematical gate, such as a Sigmoid or ReLU function, and determines whether or to what extent a neuron should “fire” based on the input it receives. After the data has passed through these transformations, the output layer produces the final prediction or classification.

As used herein a “multi-output feedforward neural network” (“MOFN”) is a type of artificial neural network architecture designed to predict multiple target variables simultaneously from a single set of inputs. These networks generally share the same basic structure as standard feedforward models, but they differ in the way the network is organized at the final stage. The network begins with an input layer that receives the raw data or features. The data is then processed through one or more hidden layers made up of neurons that apply activation functions, such as ReLU, to extract patterns and useful features from the input.

Rather than ending in a single final output layer, the network branches into multiple output layers, or heads, with each head dedicated to a particular task. For example, one head may use a Softmax activation function for classification, while another head may use a Linear activation function for regression. Since the network is designed to accomplish multiple objectives, each output is typically associated with its own loss function, such as Cross-Entropy for label prediction and Mean Squared Error for coordinate prediction.

To train the network as a unified system, the separate loss values are combined into a single global loss, often by using a weighted sum. The optimizer then minimizes that combined value so that the network can learn all the tasks together.

As used herein, “neural networks” or “artificial neural networks” refer to a specific class of machine learning algorithms. Artificial neural networks are modeled on the human brain, in which thousands or millions of processing nodes are interconnected and organized into layers.

In an artificial neural network, cells, or nodes, are connected, with each cell processing inputs and producing an output that is sent to other neurons. Labeled data moves through the nodes, or cells, with each cell performing a different function. In a neural network trained to identify whether a picture contains a cat or not, the different nodes would assess the information and arrive at an output that indicates whether a picture features a cat.

As used herein, “deep learning” refers to neural networks with many layers. The layered network can process extensive amounts of data and determine the “weight” of each link in the network. For example, in an image recognition system, some layers of the neural network might detect individual features of a face, like eyes, nose, or mouth, while another layer would be able to tell whether those features appear in a way that indicates a face. Likewise, in certain embodiments, deep learning networks may differentiate abnormal trends in monitored vital signs from normal trends.

While the machine learning model used in embodiments of the disclosure is an artificial neural network, it will be understood the machine learning model may take other forms. In embodiments, the learning model may implement linear regression, logistic regression, supervised learning, unsupervised learning, or other suitable machine learning approaches.

As used herein, the term “training set,” in the context herein refers to a collection of labeled data used to teach the model how to recognize patterns and features. This dataset includes input data, such as patient data (e.g., medical records, vital signs, etc.), along with corresponding labels or annotations that indicate the desired output. During training, the model/s used herein processes the data and adjust one or more internal parameters to minimize the difference between its predictions and the actual labels. This process enables the model to learn and generalize from the data, improving its accuracy in recognizing similar patterns in new, unseen data. Additional terms similar to “training set” may include “training data,” or “training material” which refer to any data in any form used as the whole or part of a training set. The models presented herein may be updated with one or more additional training sets from an initial training set.

As used herein, the term “vital signs” refers to objective measurements of the most essential life-sustaining functions of a body. Vital signs provide important information about the overall physical condition of a patient, allow healthcare providers to establish a baseline for comparison, and can serve as early warning indicators of medical problems or clinical deterioration.

Medical professionals routinely monitor four primary vital signs. Body temperature reflects the body's ability to maintain core heat, with a typical healthy adult range of about 97.8 °F to 99 °F, or 36.5 °C to 37.2 °C. Pulse rate, or heart rate, measures the number of heartbeats per minute and provides information about heart rhythm and strength, with a normal adult range of about 60 to 100 beats per minute. Respiration rate measures the number of breaths taken per minute, usually at rest, and is commonly about 12 to 16 breaths per minute for adults, although some references extend the normal range up to 20. Blood pressure measures the force of blood against artery walls and is recorded as systolic pressure during a heartbeat over diastolic pressure while the heart rests, with a normal reading generally considered less than 120/80 mm Hg.

In certain clinical environments, such as emergency departments, intensive care units, acute care settings, or diabetic care, additional measurements may also be treated as vital signs. Oxygen saturation, or SpO₂, is often referred to as the fifth vital sign and measures the amount of oxygen circulating in the blood, typically using a pulse oximeter. Pain may also be assessed systematically as a vital sign, although it is subjective rather than objective like the traditional four. Blood glucose may be monitored as an additional vital measurement, particularly in acute care or when managing patients with diabetes.

Vital signs are important since they support clinical decision-making across many stages of care. In triage, they help determine the urgency of patient needs. During treatment or recovery, tracking vital signs helps clinicians evaluate whether a patient is improving, stabilizing, or worsening. Significant changes in vital signs may also provide early detection of serious conditions, including cardiac arrest, sepsis, or other forms of clinical deterioration.

As used herein the term “Electronic Health Records” or “Electronic Health Record Systems” (“EHR”) refers to comprehensive digital versions of the traditional paper medical chart kept for patients. EHR records differ from older Electronic Medical Records, which were often confined to a single medical practice because EHRs are designed to support interoperability. This means that patient information can be securely shared among multiple healthcare organizations, including specialists, laboratories, hospitals, and other care providers.

EHRs provide a longitudinal view of a patient's health by combining many categories of information into a single digital record. They may include demographic information, such as a patient's name, age, gender, contact information, and insurance details. They also store clinical history, including prior illnesses, surgeries, family history, and lifestyle factors such as smoking status. In addition, EHRs maintain medical data such as diagnoses, treatment plans, progress notes, and immunization dates, as well as medication and allergy information, including current and past prescriptions, dosages, and documented drug interactions or allergy risks.

EHR systems also include objective health measurements and diagnostic information. Vital signs, such as heart rate, blood pressure, temperature, and body mass index, may be recorded in the EHR. Test results may also be integrated, including laboratory reports such as blood tests or cultures and radiology information such as X-rays, MRI scans, and CT scans. Beyond clinical content, EHRs often include administrative and billing data, such as appointment schedules, billing codes, and insurance verification records.

EHR platforms are developed by specialized healthcare technology vendors.

Large hospitals and integrated health systems often use major enterprise platforms such as

Epic Systems, Oracle Health, formerly Cerner, or MEDITECH. Smaller or ambulatory practices may use systems such as athenahealth, eClinicalWorks, NextGen Healthcare, or Practice Fusion.

In embodiments of the disclosure, patient data accessed from one or more EHR systems may be used as input for the determination of one or more acceptable ranges for one or more tracked vital signs.

As used herein the term “medical history” refers to a comprehensive written or digital record of past and present health for a person. It provides a foundation for clinical decision-making by helping healthcare providers identify patterns, evaluate future health risks, and develop accurate diagnoses. When maintained in an Electronic Health Record, the medical history can serve as a longitudinal account of patient health and may be securely shared among different providers to support continuity of care.

A complete medical history usually includes several categories of information. The chief complaint identifies the primary reason for seeking care at that moment, while the history of present illness provides a chronological description of current symptoms, including onset, severity, and any factors that make the symptoms better or worse. The past medical history records prior illnesses, chronic conditions such as diabetes or hypertension, hospitalizations, and significant childhood illnesses. Surgical history documents previous operations or procedures, including any complications involving anesthesia or healing.

Medication history and allergy information are also important parts of the record. The medication history lists current and past medications, dosages, vitamins, and herbal supplements. The allergies section documents adverse reactions to medications, foods, or environmental factors. A family medical history captures health information about close biological relatives and helps identify hereditary risks for diseases such as cancer or heart disease. A social history records lifestyle and contextual factors, including occupation, living situation, smoking, alcohol use, exercise habits, and similar information. An immunization record provides a history of vaccines received.

Maintaining an accurate medical history helps prevent clinical errors, supports risk assessment, and improves the efficiency of care. For example, it can help clinicians avoid prescribing medications that may interact dangerously with existing medications. It can also reveal predispositions to certain diseases based on family history or social factors. When kept current, the medical history allows the patient and provider to spend more time addressing present medical concerns rather than filling gaps in background information.

In embodiments of the disclosure a medical history for a patient may be obtained from one or more EHR systems may be presented into the input layer of a MOFN for the purposes of providing personalized patient-specific acceptable vital sign ranges (risk-ranges).

### ABBREVIATIONS

ADC—Analog-to-Digital Converter
ASCII—American Standard Code for Information Interchange
CD-ROM—Compact Disc Read-Only Memory
DRAM—Dynamic Random Access Memory
DVD—Digital Versatile Disc
ECG—Electrocardiogram
ED—Emergency Department
EEPROM—Electronically Erasable Programmable Read-Only Memory
EHR—Electronic Health Record/Electronic Health Record System
EKG—Electrocardiogram
EPROM—Erasable Programmable Read-Only Memory
FFNN—Feedforward Neural Network
ICU—Intensive Care Unit
eICU—Electronic Intensive Care Unit
MOFN—Multi-Output Feedforward Neural Network
PPG—Photoplethysmography
PROM—Programmable Read-Only Memory
RAM—Random Access Memory
ReLU—Rectified Linear Unit
ROM—Read-Only Memory
SpO₂—Oxygen Saturation
UVA—University of Virginia

Disclosed herein is a risk range generation system that dynamically adjusts green/yellow/red alert thresholds for each vital sign, accounting for predicted patient-specific variability. Green stands for Normal; Yellow stands for Caution and Red stands for Critical. The risk range generation system will provide alerts during consistent abnormal readings (e.g., 3 consecutive data points in yellow or red zone) or when crossing 2 zones (e.g., green to red).

Implemented in embodiments are data preprocessing pipelines, including encoding, scaling, and model inference, to enable real-time prediction and classification of patient vital signs into risk zones (green, yellow, red) based on the predicted ranges set by the model for each vital sign.

In embodiments presented herein, the implemented models may be validated against hypothetical simulated ED patients with varying pathologies demonstrating acute deterioration in males and females of varying ages. The varying pathologies include, but are not limited to: anaphylaxis, asthma, pulmonary embolism, heart attack with cardiogenic shock, pneumothorax, heart failure, mesenteric ischemia with sepsis, hypertensive emergency, pneumonia, dehydration, cocaine intoxication, narcotic overdose, ruptured abdominal aortic aneurysm.

In additional embodiments a medical history (e.g. a patient's “problem list”) and medications are presented into a trained Feedforward Neural Network to improve and personalize alarm thresholds for patients.

Further embodiments may use time-series data as a “training set” to train a neural network model that can identify vital sign trends over time and predict patient deterioration. In embodiments, the time series dataset includes approximately 1,439,576 patients (de-identified) presenting to the UVA emergency department from 2022-2025 and was obtained via established partnerships in Data Analytics at UVA Health.

In embodiments, the personalized threshold and vital sign trend models are validated with continuous real-time (de-identified) data to optimize sensitivity and specificity with feedback from clinicians in real time to ensure face validity.

Embodiments may further include integration with hospital Electronic Health Record (EHR) systems, namely EPIC, for seamless monitoring and alerts.

Further embodiments of the disclosed system can be deployed in remote hospitals and rural health centers where continuous monitoring resources are limited.

The disclosed system can be integrated into Intensive Care Units (ICUs), including both Electronic Intensive Care Units (eICUs) and traditional ICUs, to enhance remote monitoring and reduce workload on critical care staff, improving patient outcomes and workflow efficiency.

One or more disclosed embodiments can be rapidly deployed for disaster response, enabling mobile monitoring of large numbers of patients in mass casualty events, field hospitals, or quarantine zones.

Disclosed embodiments can be expanded to global health initiatives, supporting healthcare delivery in low-resource settings and regions affected by pandemics or other public health crises.

### Neural Network Description

### System Overview

In some embodiments, a physiological monitoring and reporting system generates personalized monitoring thresholds for a patient by using a trained machine learning model and applies the personalized monitoring thresholds to detect potential patient deterioration. The system includes a data preprocessing module, a neural network inference module, a threshold generation module, and an alert detection module. These modules cooperate to receive patient-specific information, predict individualized physiological parameters, generate risk zones for monitored physiological measurements, and identify alert conditions from sequences of incoming measurements (e.g., from one or more tracked vital signs).

### Input Data and Preprocessing Module

The data preprocessing module receives a patient-specific input vector that includes age, weight, gender, baseline heart rate, baseline oxygen saturation, baseline systolic blood pressure, and baseline diastolic blood pressure. In some embodiments, categorical data, such as gender, is converted into a numerical representation using label encoding.

### Feature Scaling

The input features are then standardized based on a mean and a standard deviation derived from a training dataset. This standardization may take the form:

$${x}^{′} = \frac{x - μ}{σ}$$

Where, μ is the mean of the feature from the training dataset and σ is the standard deviation of the feature.

### Training Only Output Normalization

During training, target physiological measurements may also be normalized to promote stable convergence of the neural network and to maintain consistent scaling among output values. Thus, in embodiments, target vital signs (received from one or more sensors reading from a patient) may be normalized in the form of:

$${y}^{′} = \frac{y - {μ}_{y}}{{σ}_{y}}$$

### Neural Network Model Architecture

The neural network inference module may implement a multi-output feedforward neural network (MOFN). In some embodiments, the neural network includes an input layer configured to receive seven input features, a first hidden layer having thirty-two neurons and a ReLU activation function, a second hidden layer having sixteen neurons and a ReLU activation function, a third hidden layer having eight neurons and a ReLU activation function, and an output layer having eight neurons and a linear activation function.

### Output Representation

The output layer produces an output vector that includes predicted patient-specific expected values for heart rate, oxygen saturation, systolic blood pressure, and diastolic blood pressure, and predicted variability parameters for heart rate, oxygen saturation, systolic blood pressure, and diastolic blood pressure. In certain embodiments, an output vector o∈ [symbol] is structured as:

O=[μ{HR}, μ{SPO2}, μ{SysBP}, μ{DiaBP}, σ{HR}, σ{SpO₂}, σ{SysBP}, σ{DiaBP}]

where μ represents patient-specific values and σ represents predicted variability parameters.

### Training Procedure

In some embodiments, the neural network is trained using supervised learning. Training data may be divided into training, validation, and test sets. Optimization may be performed using an Adam optimizer, and a regression loss, such as mean squared error, may be used as a loss function. Early stopping may be applied based on validation loss.

### Variability Parameter Handling

In some embodiments, variability outputs may be constrained to positive values by applying a modulus transformation, absolute value, or another non-negative transformation. The variability parameters represent patient-specific tolerance ranges used by the threshold generation module.

### Inference and Parameter Reconstruction

For a new patient, the system encodes and scales the input features and passes the resulting feature vector through the trained neural network. The system then rescales the outputs to reconstruct patient-specific physiological expectations and corresponding variability parameters. Thus, in embodiments, the output rescaling may take the form of:

μ=μ′·σ_{y}+μ_{y}

σ=|σ′·σ_{y}

The reconstructed parameters are used to generate personalized thresholds for monitored physiological measurements rather than static population-based cutoffs.

### Threshold Generation Module

The threshold generation module computes individualized risk zones from the predicted expected values and predicted variability parameters. In some embodiments, the

Lower Bound=μ−k·σ

Upper Bound=μ+k·σ

thresholds are determined using a predefined multiplier applied to a predicted variability parameter. For example, a general form for each vital may take the form of:
where k is a predefined multiplier.

### Vital-Specific Threshold Definitions

For heart rate, the system may define a green zone using a range of plus or minus one standard deviation, a yellow zone using a range of plus or minus two standard deviations, and a red zone using a range of plus or minus three standard deviations.

For oxygen saturation, the system may define a green zone using a range of plus or minus 1.5 standard deviations, clipped to a range from 0 to 100, a yellow zone using a lower extended range, and a red zone for values below the lower yellow bound.

For systolic blood pressure and diastolic blood pressure, the system may define a green zone using a range of plus or minus 1.5 standard deviations, a yellow zone using a range of plus or minus 2.5 standard deviations, and a red zone using a range of plus or minus 3.5 standard deviations.

Thus, for the above examples (extendible to those not listed herein), the thresholds define individualized risk zones rather than static population cutoffs.

### Zone Classification Module

The zone classification module classifies each incoming measurement at each timepoint as green, yellow, or red. A green classification indicates that a measurement is within an expected range, a yellow classification indicates an intermediate deviation, and a red classification indicates that the measurement is outside acceptable bounds. Classification may be performed independently for each monitored physiological measurement and for each timepoint.

### Alert Detection Module

The alert detection module analyzes temporal sequences of classifications. In some embodiments, the system evaluates sliding windows of three measurements across time-series data. The system may trigger an alert when qualifying patterns are detected, including three consecutive red measurements, three consecutive yellow measurements, a yellow-to-yellow-to-red progression, or a monotonic escalation such as green-to-yellow-to-red. The alert detection module may trigger an alert upon the first detection of a qualifying pattern for one or more monitored physiological measurements.

### Medical Device Embodiment

One or more embodiments of the above described MOFN may be present in a medical device configured to physically measure one or more vital signs, generating a vital sign input, and then present the vital sign input into the input layer of one or more embodiments of the MOFN presented herein. The medical device may contain all the necessary components of a computer as above discussed and one or more analog-to-digital conversion devices used to measure and convert vital signs. Several non-limiting example vital sign measurement devices are below presented.

Cardiac and respiratory monitors include several types of devices that acquire physiological signals and convert them into digital information for analysis. Electrocardiogram devices, also referred to as ECG or EKG devices, use electrodes to detect electrical biopotentials produced by the heart. Since these signals are very small, an analog front end, such as an ADS1293-type component, may amplify the analog signals before the signals are converted into digital data for heart rhythm analysis.

Pulse oximeters measure blood oxygen saturation, or SpO₂, by passing red and infrared light through tissue. A photodetector converts the detected light intensity into an analog current, and that current is then digitized, often using a 14-bit or 24-bit analog-to-digital converter, so that oxygen levels can be calculated.

Digital stethoscopes operate by amplifying internal body sounds, such as heart, lung, or bowel sounds, and using analog-to-digital conversion to transform acoustic vibrations into digital audio that can be transmitted to clinicians or analyzed for abnormalities.

Temperature and pressure devices also rely on sensors that convert physiological changes into measurable electrical signals. Digital thermometers commonly use a thermistor, which is a resistor having resistance that changes based on temperature. The analog change in resistance is converted into a digital temperature reading. Certain medical-grade temperature sensing components, such as MAX30205-type components, may be configured to provide high-accuracy temperature measurements.

Automatic blood pressure cuffs typically use an oscillometric method to detect arterial vibrations during cuff inflation or deflation. A pressure sensor captures the analog pressure variations, and the captured signals are digitized and processed by algorithms to determine systolic and diastolic blood pressure.

Portable and wearable sensors extend these monitoring functions into continuous or ambulatory settings. Continuous monitoring patches are small adhesive wearable devices that may integrate multiple sensors, such as ECG sensors, temperature sensors, and accelerometers, into a compact system for tracking vital signs over time. Smartwatches and other consumer wearable devices may use optical sensors to perform photoplethysmography, or PPG. These devices digitize reflected light signals to monitor heart rate and, in some cases, detect irregular rhythms such as atrial fibrillation.

Therefore, embodiments of the disclosure may include one or more medical devices associated, attached, placed, or otherwise interfacing with a patient to measure one or more vital signs and present the measurements as input into one or more embodiments of the model presented herein.

Further details of operations and aspects of the disclosed embodiments are now presented with further reference to the Figures which provide non-limiting illustrations of the disclosure.

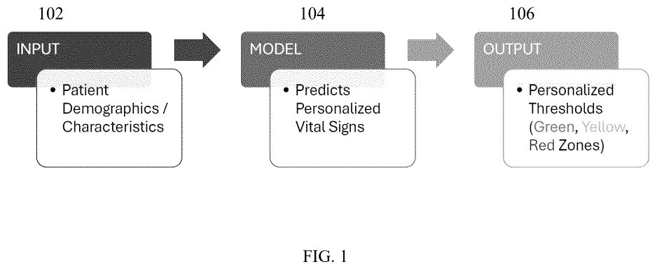

FIG. 1 illustrates a high-level schematic of an embodiment of the neural network model disclosed herein. Input values 102 such as patient demographics and characteristics, including information obtained from one or more EHR systems are input into a MFON Model 104 which generates an output 106 in the form of personalized thresholds.

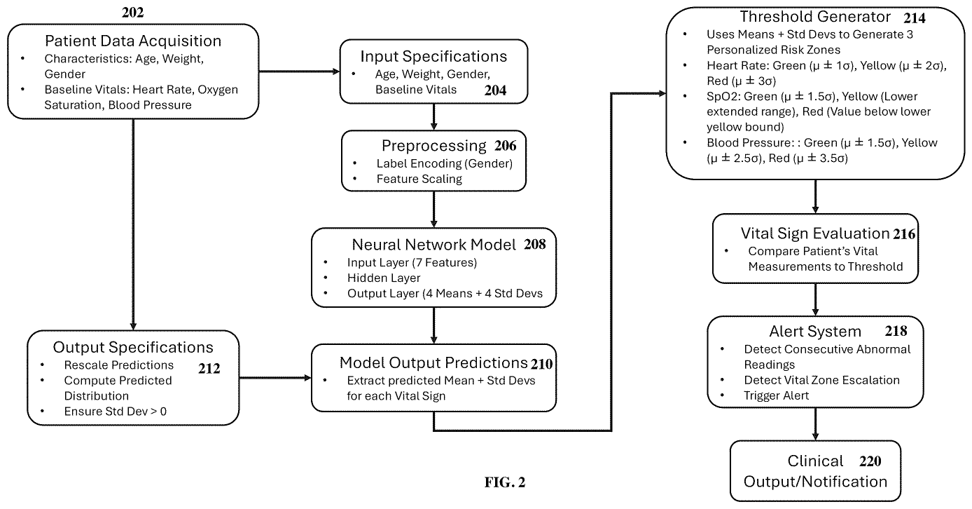

FIG. 2 provides a flow chart of an embodiment of a neural network model disclosed herein. The process begins with patient data acquisition 202, in which patient characteristics such as age, weight, and gender are collected along with baseline vital signs, including heart rate, oxygen saturation, and blood pressure. The data are then input into the model 204. The acquired data is then preprocessed 206, for example, by label encoding gender and applying feature scaling so that the information can be provided to a neural network model in a suitable numerical format.

The neural network model 208 receives the input features such as those corresponding to age, weight, gender, and baseline vital signs. The model includes an input layer, at least one hidden layer, and an output layer configured to generate predicted values for the patient. The model output layer includes four predicted mean values and four predicted standard deviation values, corresponding to the monitored vital signs. The model output predictions 210 includes extracting predicted means and standard deviations for each vital sign tracked.

Output specifications 212 may incorporate acquired patent data obtained from patient data acquisition step 202. Predicted values are rescaled, predicted distributions are computed, and the system ensures that each standard deviation value is greater than zero. The output specifications 212 may then be incorporated into the model output predictions 210 to provide normalized data to a threshold generator 214.

The predicted means and standard deviations are then used by a threshold generator 214 to create three personalized risk zones for each vital sign. For heart rate, the zones are based on ranges around the predicted mean using one, two, and three standard deviations. For oxygen saturation, the green zone is based on approximately 1.5 standard deviations around the mean, with a lower extended yellow range and a red zone for values below the lower yellow bound. For blood pressure, the zones are based on approximately 1.5, 2.5, and 3.5 standard deviations around the predicted mean.

The workflow also includes vital sign evaluation 216. Patient vital measurements are then compared against the personalized thresholds. An alert system 218 detects consecutive abnormal readings or escalation among vital sign zones and triggers a clinical output or notification 220 when an alert condition is identified. Such output/notification may include triggering warnings or alerts on one or more displays, sounding one or more alarm notifications, or sending messages (e.g., email, text, automated phone call) to appropriate clinical staff and/or the patient.

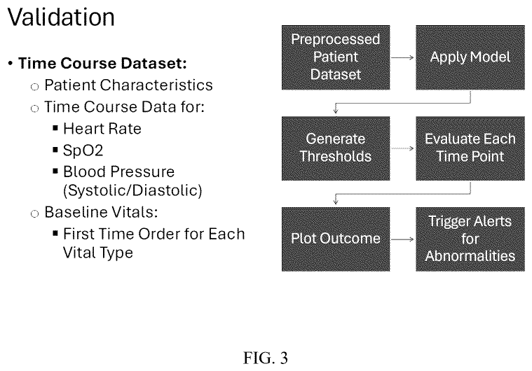

FIG. 3 illustrates a validation process for a neural network model configured to generate personalized vital sign thresholds. In the illustrated process, patient-specific information, including demographic data and prior physiological measurements, may be used by the neural network model to predict individualized threshold values for monitored vital signs, such as heart rate, oxygen saturation, systolic blood pressure, and diastolic blood pressure.

As shown in FIG. 3, the model may be validated using validation data and clinical feedback to confirm or optimize the performance of the model. In some implementations, the validation data may include simulated emergency department patient data representing different pathologies and demographic characteristics, such as variations in age and gender. The model may also be evaluated using continuous real-time de-identified patient data. Feedback from clinicians may be used to refine model performance, including sensitivity and specificity, and to confirm that the generated personalized thresholds and trend-based predictions are clinically reasonable for patient monitoring.

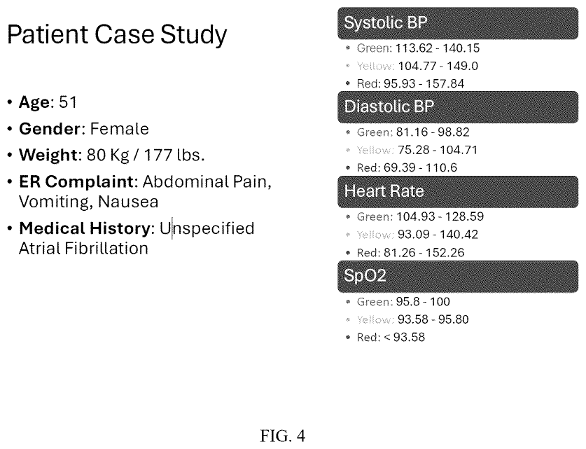

FIG. 4 illustrates patient information for a patient case study generated from the disclosed neural network model. In the case study, patient-specific characteristics and baseline physiological measurements are used as input data for the model. The patient information may include demographic characteristics, such as age, weight, and gender, together with baseline vital signs, such as heart rate, oxygen saturation, systolic blood pressure, and diastolic blood pressure.

The information shown in FIG. 4 provides the patient-specific context used to generate individualized model outputs. Rather than applying static population-based thresholds, the model uses the patient information to predict expected physiological values and patient-specific variability parameters. These predicted values and variability parameters may then be used to establish personalized risk zones for monitored vital signs and to support later evaluation of systolic blood pressure, diastolic blood pressure, pulse, and oxygen saturation over time.

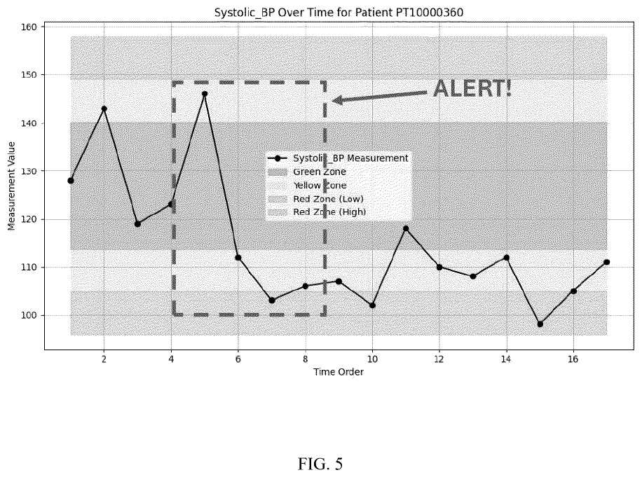

FIG. 5 illustrates output information for a patient case study generated by the disclosed neural network model. In the illustrated case study, the model uses patient-specific input information to generate individualized threshold ranges for monitored vital signs, including systolic blood pressure, diastolic blood pressure, heart rate, and oxygen saturation. The thresholds are organized into personalized risk zones, such as green, yellow, and red zones, which represent normal, caution, and critical ranges for the particular patient rather than static population-based limits.

As shown in FIG. 5, the generated thresholds may include individualized ranges for systolic blood pressure, diastolic blood pressure, and heart rate, as well as oxygen saturation ranges in which a lower oxygen saturation value may indicate a higher-risk condition. The figure further indicates that alerts may be generated when monitored vital sign measurements fall within abnormal risk zones or otherwise satisfy alert conditions. In this manner, FIG. 5 demonstrates how the model output may be presented for a patient case study and used to support real-time monitoring, personalized risk classification, and clinical notification.

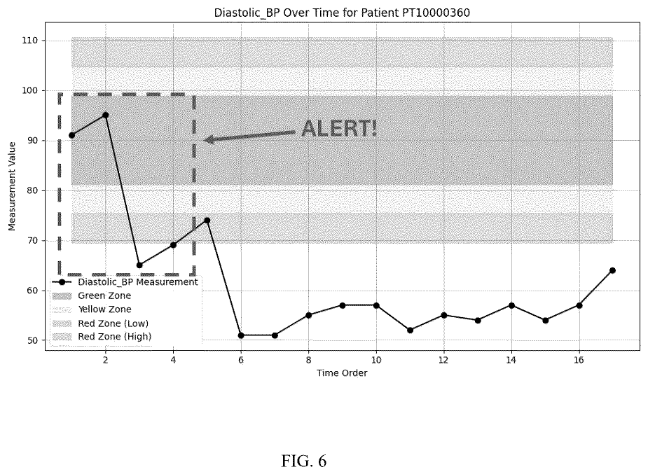

FIG. 6 illustrates the alerting aspect of the patient case study generated using the disclosed neural network model. In the illustrated example, individualized threshold ranges for the patient are applied to monitored vital sign measurements over time so that each measurement can be evaluated against personalized green, yellow, and red risk zones.

As shown in FIG. 6, the system may identify abnormal physiological conditions when one or more vital sign measurements fall outside an expected patient-specific range or progress into higher-risk zones. The figure demonstrates that alerts may be triggered based on detected abnormalities, such as measurements entering caution or critical ranges, repeated abnormal readings, or escalation across risk zones. In this manner, FIG. 6 illustrates how the personalized threshold outputs can be used in a clinical monitoring workflow to identify potential patient deterioration and generate notifications for clinical attention.

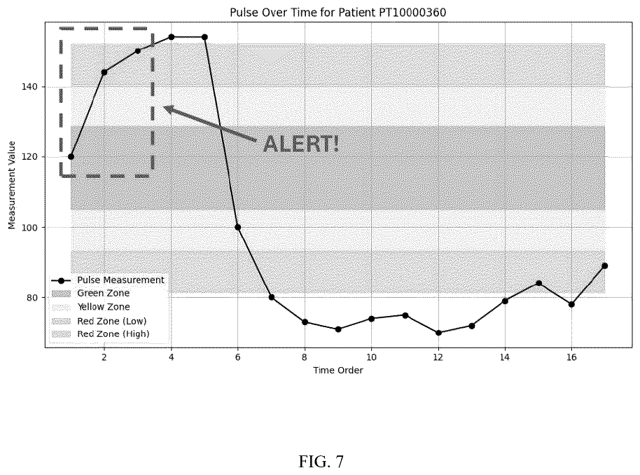

FIG. 7 illustrates pulse measurements over time for a patient case study generated from the disclosed neural network model. In the illustrated case study, patient-specific information is processed to generate individualized heart rate thresholds, and the patient pulse measurements are plotted over time relative to those personalized risk zones.

As shown in FIG. 7, the pulse values may be classified into green, yellow, or red zones based on the personalized threshold ranges generated for the patient. The green zone represents a normal pulse range for the patient, the yellow zone represents a cautionary range, and the red zone represents a critical range. By displaying pulse measurements over time against these patient-specific zones, FIG. 7 shows how the system can identify abnormal heart rate patterns, detect escalation in risk classification, and support alert generation when consistent abnormal readings or zone-crossing events occur.

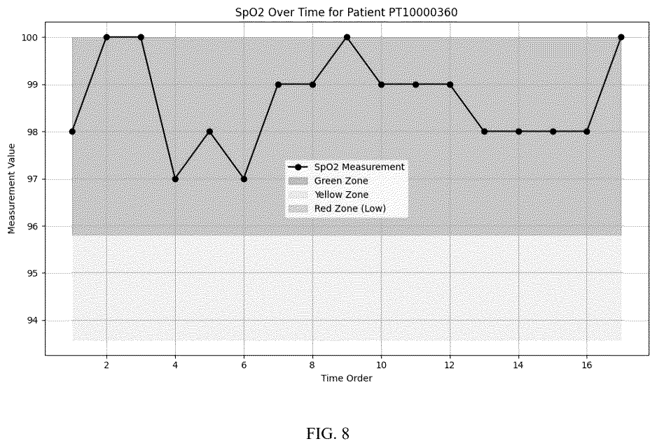

FIG. 8 illustrates oxygen saturation measurements over time for a patient case study generated from the disclosed neural network model. In the illustrated case study, patient-specific information is processed to generate individualized oxygen saturation thresholds, and the patient oxygen saturation measurements are plotted over time relative to personalized risk zones.

As shown in FIG. 8, oxygen saturation values may be classified into green, yellow, or red zones based on the personalized threshold ranges generated for the patient. The green zone represents a normal oxygen saturation range for the patient, the yellow zone represents a cautionary range below the green zone, and the red zone represents a critical range below the yellow zone. By displaying oxygen saturation measurements over time against these individualized zones, FIG. 8 shows how the system can identify abnormal oxygenation patterns, detect movement into lower-risk or higher-risk classifications, and support alert generation when consistent abnormal readings or escalation events occur.

### General Disclosure

While various embodiments of the present disclosure are described herein, it will be understood by those skilled in the art that such embodiments are provided by way of example only. It will be understood by those skilled in the art that numerous modifications and changes to, and variations and equivalent substitutions of, the embodiments described herein can be made without departing from the scope of the disclosure. It is understood that various alternatives to the embodiments described herein may be employed in practicing the disclosure, and modifications may be made to adapt a particular structure or material to the teachings of the disclosure. It is also understood that every embodiment of the disclosure may optionally be combined with any one or more of the other embodiments described herein which are consistent with that embodiment.

Where elements are presented in list format (e.g., in a Markush group), it is understood that each possible subgroup of the elements is also disclosed, and any one or more elements can be removed from the list or group.

It is also understood that, unless clearly indicated to the contrary, in any method described or claimed herein that includes more than one act or step, the order of the acts or steps of the method is not necessarily limited to the order in which the acts or steps of the method are recited, but the disclosure encompasses embodiments in which the order is so limited.

It is further understood that, in general, where an embodiment in the description or the claims is referred to as comprising one or more features, the disclosure also encompasses embodiments that consist of, or consist essentially of, such feature(s).

It is also understood that any embodiment of the disclosure, e.g., any embodiment found within the prior art, can be explicitly excluded from the claims, regardless of whether the specific exclusion is recited in the specification.

Headings are included herein for reference and to aid in locating certain sections. Headings are not intended to limit the scope of the embodiments and concepts described in the sections under those headings, and those embodiments and concepts may have applicability in other sections throughout the entire disclosure.

All patent literature and all non-patent literature cited herein are incorporated herein by reference in their entirety to the same extent as if each patent literature or non-patent literature were specifically and individually indicated to be incorporated herein by reference in its entirety.

Unless otherwise defined, all technical and scientific terms used herein have the same meaning as commonly understood by one of ordinary skill in the art to which this disclosure belongs.

Where a range of values is provided, it is understood that each intervening value between the upper and lower limit of that range and any other stated or intervening value in that stated range is encompassed within the disclosure. The upper and lower limits of these smaller ranges may independently be included in the smaller ranges is also encompassed within the disclosure, subject to any specifically excluded limit in the stated range. Where the stated range includes one or both of the limits, ranges excluding both of those included limits are also included in the disclosure.

The articles “a” and “an” as used herein and in the appended claims are used herein to refer to one or to more than one (i.e., to at least one) of the grammatical object of the article unless the context clearly indicates otherwise. By way of example, “an element” means one element or more than one element.

The term “exemplary” as used herein means “serving as an example, instance or illustration”. Any embodiment or feature characterized herein as “exemplary” is not necessarily to be construed as preferred or advantageous over other embodiments or features.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either” “one of,” “only one of,” or “exactly one of.”

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a nonlimiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, in certain methods described herein that include more than one step or act, the order of the steps or acts of the method is not necessarily limited to the order in which the steps or acts of the method are recited unless the context indicates otherwise.

The term “about” or “approximately” means an acceptable error for a particular value as determined by one of ordinary skill in the art, which depends in part on how the value is measured or determined. In certain embodiments, the term “about” or “approximately” means within one standard deviation. In some embodiments, when no particular margin of error (e.g., a standard deviation to a mean value given in a chart or table of data) is recited, the term “about” or “approximately” means that range which would encompass the recited value and the range which would be included by rounding up or down to the recited value as well, taking into account significant figures. In certain embodiments, the term “about” or “approximately” means within 10% or 5% of the specified value. Whenever the term “about” or “approximately” precedes the first numerical value in a series of two or more numerical values or in a series of two or more ranges of numerical values, the term “about” or “approximately” applies to each one of the numerical values in that series of numerical values or in that series of ranges of numerical values.

Whenever the term “at least” or “greater than” precedes the first numerical value in a series of two or more numerical values, the term “at least” or “greater than” applies to each one of the numerical values in that series of numerical values.

Whenever the term “no more than” or “less than” precedes the first numerical value in a series of two or more numerical values, the term “no more than” or “less than” applies to each one of the numerical values in that series of numerical values.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein without any additional undue experimentation. It being understood that various changes may be made in the function and arrangement of elements described in an exemplary embodiment without departing from the scope of the various embodiments as set forth in the appended claims.

Since certain changes may be made in the above-described disclosure, without departing from the spirit and scope of the disclosure herein involved, it is intended that all of the subject matter of the above description shown in the accompanying drawings shall be interpreted merely as examples illustrating the inventive concept herein and shall not be construed as limiting the disclosure.

In addition, it is understood that any functional language used in any claims shall not be construed as “means-plus-function” language under 35 U.S.C. § 112(f), unless specifically expressed as such by use of the term “means for” or “step(s) for” in a claim.

### EXAMPLES

In some illustrative embodiments, a physiological monitoring system includes one or more physiological sensors configured to generate physiological sign measurements for a patient, one or more processors communicatively coupled to the one or more physiological sensors, and one or more non-transitory computer-readable media storing instructions executable by the one or more processors. When executed by the one or more processors, the instructions cause the physiological monitoring system to receive patient-specific data for the patient, preprocess the patient-specific data to generate a patient-specific input vector, apply the patient-specific input vector to a trained neural network model, and generate, using the trained neural network model, patient-specific expected values and corresponding patient-specific variability parameters for a plurality of physiological signs. The instructions further cause the physiological monitoring system to generate individualized risk zones for each physiological sign based on the patient-specific expected values and the corresponding patient-specific variability parameters, where the patient-specific variability parameters define patient-specific tolerance ranges. The instructions further cause the physiological monitoring system to classify the physiological sign measurements relative to the individualized risk zones to generate a temporal sequence of classifications, detect a temporal pattern in the temporal sequence of classifications indicative of physiological deterioration, and generate a clinical notification in response to detecting the temporal pattern. In this manner, the physiological monitoring system can improve real-time monitoring by dynamically adapting threshold ranges based on patient-specific variability.

In some illustrative embodiments, the one or more physiological sensors include at least one of an electrocardiogram sensor, a pulse oximeter, a digital thermometer, a blood pressure sensor, a digital stethoscope, a wearable patch sensor, or an optical photoplethysmography sensor. The physiological monitoring system can be configured for use in an emergency department waiting room. The clinical notification can be generated by activating a display alert, an audible alarm, an electronic message, an automated telephone notification, or a combination thereof. The physiological monitoring system can be integrated with an Electronic Health Record system through bidirectional communication that updates the patient-specific data, the individualized risk zones, or both.

In some illustrative embodiments, the individualized risk zones are defined based on a plurality of ranges derived from the patient-specific variability parameters. The individualized risk zones can include ranges defined by one or more standard deviation-based multipliers applied to the patient-specific variability parameters. Different physiological signs of the plurality of physiological signs can be associated with different variability-based threshold definitions. In some implementations, the physiological sign measurements are processed in real time. In some implementations, the trained neural network model is configured to simultaneously output both the patient-specific expected values and the patient-specific variability parameters in a single inference pass. The clinical notification can be generated only when a plurality of consecutive measurements satisfy an alert condition.

In some illustrative embodiments, a non-transitory computer-readable medium stores instructions that, when executed by one or more processors, cause the one or more processors to perform operations for personalized physiological monitoring. The operations include receiving patient-specific data for a patient from an Electronic Health Record system and one or more physiological monitoring devices, encoding and scaling the patient-specific data to generate a patient-specific input vector, processing the patient-specific input vector using a trained multi-output feedforward neural network, and generating predicted expected values and predicted variability parameters for one or more physiological signs. The operations further include computing personalized threshold ranges for the one or more physiological signs using the predicted expected values and the predicted variability parameters, where the variability parameters define patient-specific tolerance ranges, assigning incoming physiological sign measurements to risk zones defined by the personalized threshold ranges to generate a temporal sequence of classifications, detecting a temporal alert condition based on the temporal sequence of classifications, and outputting an alert in response to detecting the temporal alert condition. In this manner, the operations can improve monitoring accuracy by dynamically adapting threshold ranges to patient-specific variability.

In some illustrative embodiments, the temporal alert condition includes a three-measurement sliding window. The temporal alert condition can include a yellow-to-yellow-to-red progression, a green-to-yellow-to-red progression, or another escalation pattern among risk zones. The operations can further include updating, validating, or updating and validating the trained multi-output feedforward neural network based on validation loss, clinician feedback, additional de-identified patient data, or a combination thereof.

In some illustrative embodiments, a computer-implemented method for personalized physiological monitoring of a patient includes receiving, by one or more processors, patient-specific data for the patient, the patient-specific data including demographic data and baseline physiological sign measurements, and preprocessing, by the one or more processors, the patient-specific data to generate a patient-specific input vector. The method further includes applying, by the one or more processors, the patient-specific input vector to a trained neural network model and generating, by the trained neural network model, patient-specific expected values and patient-specific variability parameters for a plurality of physiological signs. The method further includes generating, by the one or more processors, individualized risk zones for each physiological sign of the plurality of physiological signs based on a patient-specific expected value and a corresponding patient-specific variability parameter, where the variability parameters define patient-specific tolerance ranges. The method further includes receiving, by the one or more processors, physiological sign measurements for the patient from one or more physiological sensors, classifying, by the one or more processors, each physiological sign measurement relative to the individualized risk zones to generate a temporal sequence of zone classifications, detecting a temporal pattern in the temporal sequence of zone classifications indicative of physiological deterioration, and generating, by the one or more processors, a clinical notification in response to detecting an alert condition within the temporal pattern.

Finally, the written description uses examples to disclose the disclosure, including the best mode, and also to enable any person skilled in the art to practice the disclosure, including making and using any devices or systems and performing any incorporated methods. The patentable scope of the disclosure is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

What is claimed is:

1. A physiological monitoring system, comprising:
one or more physiological sensors configured to generate vital sign measurements for a patient;
one or more processors communicatively coupled to the one or more physiological sensors; and,
one or more non-transitory computer-readable media storing instructions that, when executed by the one or more processors, cause the physiological monitoring system to:
receive patient-specific data for the patient, preprocess the patient-specific data;
preprocess the patient-specific data to generate a patient-specific input vector;
apply the preprocessed patient-specific data to a trained neural network model;
generate, using the trained neural network model, patient-specific expected values and corresponding patient-specific variability parameters for a plurality of physiological signs;
generate individualized risk zones for each physiological sign based on the patient-specific expected values and the corresponding patient-specific variability parameters, wherein the variability parameters define patient-specific tolerance ranges;
classify the physiological sign measurements relative to the individualized risk zones to generate a temporal sequence of classifications;
detect a temporal pattern in the temporal sequence of classifications indicative of physiological deterioration; and,
generate a clinical notification in response to detecting the temporal pattern,
wherein the physiological monitoring system improves real-time monitoring by dynamically adapting threshold ranges based on patient-specific variability.

2. The physiological monitoring system of claim 1, wherein the one or more physiological sensors comprise at least one of: an electrocardiogram sensor, a pulse oximeter, a digital thermometer, a blood pressure sensor, a digital stethoscope, a wearable patch sensor, or an optical photoplethysmography sensor.

3. The physiological monitoring system of claim 1, wherein the physiological monitoring system is configured for use in an emergency department waiting room.

4. The physiological monitoring system of claim 1, wherein the instructions cause the physiological monitoring system to generate the clinical notification by activating a display alert, an audible alarm, an electronic message, or an automated telephone notification.

5. The physiological monitoring system of claim 1, wherein the physiological monitoring system is integrated with an Electronic Health Record system through bidirectional communication that updates the patient-specific data and the individualized risk zones.

6. The physiological monitoring system of claim 1, wherein the individualized risk zones are defined based on a plurality of ranges derived from the patient-specific variability parameters.

7. The physiological monitoring system of claim 1, wherein the individualized risk zones comprise ranges defined by one or more standard deviation-based multipliers applied to the patient-specific variability parameters.

8. The physiological monitoring system of claim 1, wherein different physiological signs of the plurality of physiological signs are associated with different variability-based threshold definitions.

9. The physiological monitoring system of claim 1, wherein the physiological sign measurements are processed in real time.

10. The physiological monitoring system of claim 1, wherein the trained neural network model is configured to simultaneously output both the patient-specific expected values and the patient-specific variability parameters in a single inference pass.

11. The physiological monitoring system of claim 1, wherein the clinical notification is generated only when a plurality of consecutive measurements satisfy the alert condition.

12. A non-transitory computer-readable medium storing instructions that, when executed by one or more processors, cause the one or more processors to perform operations, comprising:
receiving patient-specific data for a patient from an Electronic Health Record system and one or more physiological monitoring devices;
encoding and scaling the patient-specific data to generate a patient-specific input vector;
processing the patient-specific input vector using a trained multi-output feedforward neural network;
generating predicted expected values and predicted variability parameters for one or more physiological signs;
computing personalized threshold ranges for the one or more physiological signs using the predicted expected values and the predicted variability parameters, wherein the variability parameters define patient-specific tolerance ranges;
assigning incoming physiological sign measurements to risk zones defined by the personalized threshold ranges to generate a temporal sequence of classifications;
detecting a temporal alert condition based on the temporal sequence of classifications; and,
outputting an alert in response to detecting the temporal alert condition,
wherein the operations improve monitoring accuracy by dynamically adapting threshold ranges to patient-specific variability.

13. The non-transitory computer-readable medium of claim 12, wherein the temporal alert conditions comprises a three-measurement sliding window.

14. The non-transitory computer-readable medium of claim 12, wherein the temporal alert condition comprises a yellow-to-yellow-to-red progression.

15. The non-transitory computer-readable medium of claim 12, wherein the temporal alert condition comprises a green-to-yellow-to-red progression.

16. The non-transitory computer-readable medium of claim 12, wherein the operations further comprise updating and/or validating the trained multi-output feedforward neural network based on validation loss, clinician feedback, or additional de-identified patient data.

17. A computer-implemented method for personalized physiological monitoring of a patient, comprising:
receiving, by one or more processors, patient-specific data for the patient, the patient-specific data comprising demographic data and baseline physiological sign measurements;
preprocessing, by the one or more processors, the patient-specific data to generate a patient-specific input vector;
applying, by the one or more processors, the patient-specific input vector to a trained neural network model;
generating, by the trained neural network model, patient-specific expected values and patient-specific variability parameters for a plurality of physiological signs;
generating, by the one or more processors, individualized risk zones for each physiological sign of the plurality of physiological signs based on a patient-specific expected value and a corresponding patient-specific variability parameter, wherein the variability parameters define patient-specific tolerance ranges;
receiving, by the one or more processors, physiological sign measurements for the patient from one or more physiological sensors;
classifying, by the one or more processors, each physiological sign measurement of the physiological sign measurements relative to the individualized risk zones to generate a temporal sequence of zone classifications;
detecting a temporal pattern in the temporal sequence of classifications indicative of physiological deterioration; and,
generating, by the one or more processors, a clinical notification in response to detecting an alert condition within the temporal pattern.
